(19) United States
(12) Patent Application Publication
(10) Pub. No.: US 2004/0171144 A1
(43) Pub. Date: Sep. 2, 2004

(54) Increasing sperm motility

(76) Inventors: Nancy L. Brackett, Miami Lakes, FL (US); Charles M. Lynne, Miami, FL (US); Sarmistha Basu, Santa Rosa, CA (US); Daniel Cohen, Miami Beach, FL (US)

Correspondence Address: Stanley A. Kim, Ph.D., Esq., Akerman Senterfitt, Suite 400, 222 Lakeview Avenue, West Palm Beach, FL, 33401-6183, US

(21) Appl. No.: 10/748,637

(22) Filed: Dec. 30, 2003

Related U.S. Application Data
(60) Provisional application No. 60/440,832, filed on Jan. 16, 2003.

(51) Int. Cl. C12N 5/06; C12N 5/16

(52) U.S. Cl. 435/335; 424/145.1

(57) ABSTRACT

The motility of sperm in a biological sample is increased by inactivating or reducing the biological activity of inflammatory cytokines present in the sample.

### CROSS-REFERENCE TO RELATED APPLICATIONS

The present application claims the priority of U.S. provisional patent application serial No. 60/440,832 filed Jan. 16, 2003.

### FIELD OF THE INVENTION

This invention relates generally to the fields of reproductive biology and medicine. More particularly, the invention relates to methods of improving fertility by increasing sperm motility in infertile or subfertile males including patients with spinal cord injuries (SCI).

### BACKGROUND

More than 10,000 cases of SCI occur annually in the United States. Most men with SCI have impaired fertility. Potential contributing factors to this condition are multiple and can include: a) autonomic and neuromuscular dysfunction that hampers erection and ejaculation; b) semen with low sperm motility (Brackett et al., 1996a; Brackett et al., 1996b; Sonsken and Biering-Sorensen, 1992; Ohl et al., 1992; Linsenmyer and Perkash 1991); c) leukocytospermia, i.e., the presence of white blood cells (WBC) in the semen of men with SCI (Wolff et al., 1990; Aitken et al., 1991 Kovalski et al., 1992; Aitken et al., 1994; Aird et al., 1999; Basu et al., 2002) and d) cytokines or reactive oxygen species secreted by WBCs which have a cytotoxic effect on sperm cells (Padron et al., 1997; de Lamirande and Gagnon, 1993; Rajasekaran et al., 1995; Gruschwitz et al., 1996; Naz and Evans, 1998).

The macroscopic and microscopic appearance of semen in men with SCI is abnormal. Often the semen is yellow or brown in color and contains numerous non-spermatozoon cell types, many of which are leukocytes (Wieder et al., 1999; Aird et al., 1999). According to World Health Organization criteria (WHO, 1999), concentrations of leukocytes greater than one million per milliter in the ejaculate are considered abnormal. In non-SCI populations, increased leukocytes are related to low sperm motility (Wolff et al., 1990).

The majority of the patients suffering from SCI are men in the child-rearing age group (Stover et al, 1995). A need exists for methods that improve the chances of achieving fatherhood among subjects with leukocytospermia, including men with SCI.

### SUMMARY

What has been discovered is that sperm motility can be improved in semen samples from male subjects with leukocytospermia by inactivating cytokines present in the semen of these subjects. This finding is particularly advantageous for improving the chances of paternity for men suffering from SCI, in whom leukocytospermia is prevalent.

Accordingly, the invention provides a method of increasing motility of sperm. The method includes the steps of: a) providing from a subject a biological sample containing sperm and at least one cytokine and b) contacting the biological sample with an agent that inactivates or reduces the biological activity of at least one cytokine present in the sample. The method is especially useful for subjects, including SCI patients, with conditions such as leukocytospermia causing infertility.

The biological sample can be semen, or a biological fluid produced by at least one tissue of the male reproductive tract including testis, epididymis, vas deferens, prostate, or seminal vesicles. In other embodiments, the fluid contacting the sperm can be within the female reproductive tract and can be produced by tissues including the ovaries, fallopian tubes, uterus, cervix, and vagina.

The agent that inactivates the cytokine can be an antibody that specifically binds to the cytokine or cytokine receptor. In preferred embodiments, the cytokine can be one or more inflammatory cytokines including TNFα, IL1β, and IL6. In other embodiments, the agent can be a soluble form of a cytokine receptor.

As used herein, “bind,” “binds,” or “interacts with” means that one molecule recognizes and adheres to a particular second molecule in a sample, but does not substantially recognize or adhere to other structurally unrelated molecules in the sample. Generally, a first molecule that “specifically binds” a second molecule has a binding affinity greater than about 10⁵ to 10⁶ moles/liter for that second molecule.

By reference to an “antibody that specifically binds” another molecule is meant an antibody that binds the other molecule, and displays no substantial binding to other naturally occurring proteins other than those sharing the same antigenic determinants as other molecule. The term “antibody” includes polyclonal and monoclonal antibodies as well as antibody fragments or portions of immunolglobulin molecules that can specifically bind the same antigen as the intact antibody molecule.

The term “subject,” as used herein, means a human or non-human animal, including but not limited to a mammal such as a dog, cat, horse, cow, pig, sheep, goat, chicken, primate, rat, and mouse.

Unless otherwise defined, all technical terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including any definitions will control. In addition, the particular embodiments discussed below are illustrative only and not intended to be limiting.

Other features and advantages of the invention will be apparent from the following detailed description, and from the claims.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIG. 1 is a graph showing total leukocyte count in the semen of SCI and control subjects as determined by the trypan blue exclusion method.

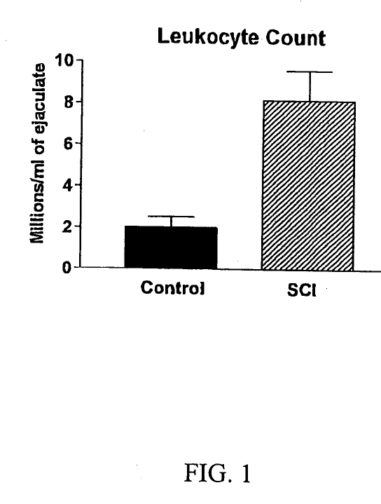

FIG. 2 is a graph depicting the percentage of CD45+ cells in SCI and control subjects determined in three gated areas based on forward and side scatter characteristics of lymphocytes, monocytes and granulocytes.

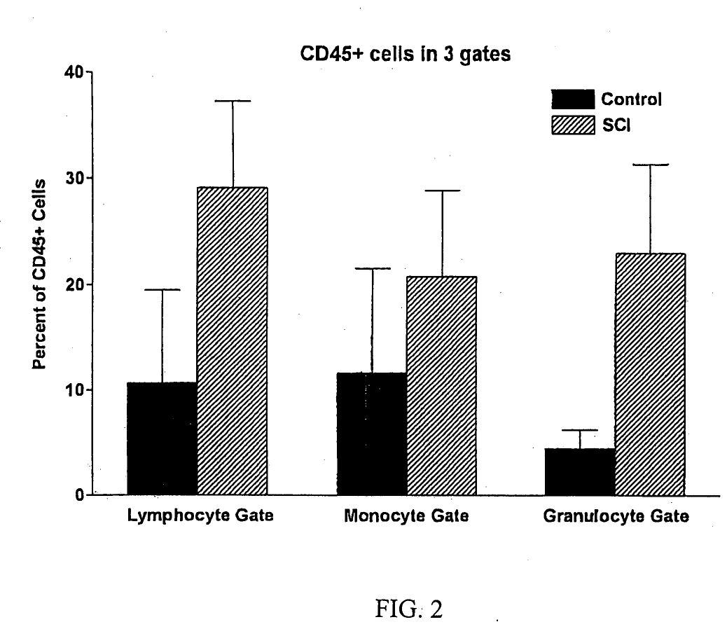

FIG. 3 is a graph showing the relative percentages of hematopoietic cell subpopulations present in semen of SCI and control subjects.

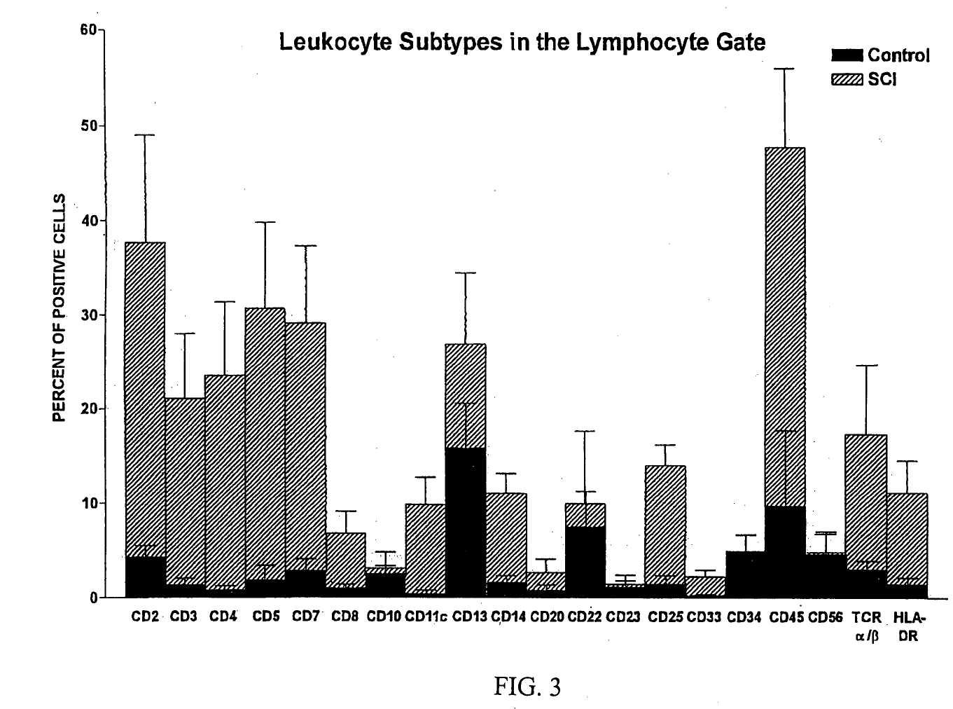

### DETAILED DESCRIPTION

The invention provides methods and compositions for increasing motility of sperm from male subjects with elevated levels of cytokines in their semen. Increased levels of cytokines in the seminal plasma can be due to the presence of activated lymphocytes, which secrete these substances into the surrounding medium. This can occur in disorders of fertility such as leukocytospermia, a condition characterized by the presence in the semen of a significantly higher concentration of granulocytes and lymphocytes than found in normal controls.

It has been shown that men with SCI have leukocytospermia. Flow cytometric analysis of leukocytes from patients with SCI demonstrated that the majority of these cells were T cells, many of which were in an activated state. Based on the knowledge that activated T cells can produce cytokines, studies were performed to screen for the levels of ten different cytokines belonging to Th1 and Th2 group, in semen samples from men with SCI and able-bodied men with no history of infertility. Results of this analysis revealed elevated levels of three inflammatory cytokines (IL1β, IL6, and TNFα) in the semen of men with SCI, relative to those of able-bodied men. Pursuant to this discovery, evidence was obtained to demonstrate that sperm motility can be increased in semen samples from men with SCI by inhibition of inflammatory cytokines present in the seminal plasma.

### Biological Methods

Methods involving conventional immunological and molecular biological techniques are described herein. Such techniques are generally known in the art and are described in detail in methodology treatises. Immunological methods (e.g., preparation of antigen-specific antibodies, immunoprecipitation and immunoblotting) are described, e.g., in Current Protocols in Immunology, ed. Coligan et al., John Wiley & Sons, New York, 1991; and Methods of Immunological Analysis, ed. Masseyeffet al., John Wiley & Sons, New York, 1992.

Molecular biological techniques are described in references such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Various techniques using polymerase chain reaction (PCR) are described, e.g., in Innis et al., PCR Protocols: A Guide to Methods and Applications, Academic Press: San Diego, 1990. Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981. Chemical synthesis of nucleic acids can be performed, for example, on commercial automated oligonucleotide synthesizers.

### Inactivating Cytokines Affecting Sperm Motility

The invention features a method of increasing motility of sperm that includes the steps of: a) providing from a subject a biological sample including sperm and at least one cytokine; and b) contacting the sample with an agent that inactivates or reduces the biological activity of at least one cytokine present in the sample.

Suitable subjects for use in the invention can be any male animal capable of producing sperm. The motile sperm can be used to impregnate any female animal capable of fertilization by sperm. The male subject can be an animal such as a mammal, e.g., a dog, cat, horse, cow, pig, sheep, goat, chicken, primate, rat, or mouse. Because the experiments presented herein relate to human subjects, a preferred subject for the methods of the invention is a human male. Particularly preferred are subjects suspected of having or at risk for developing a fertility disorder involving reduced motility of sperm, e.g., a man having leukocytospermia, based on clinical findings or other diagnostic test results. The method is especially well suited to improving motility of sperm from men with SCI, many of whom exhibit leukocytospermia.

The method of the invention includes a step of providing a biological sample including sperm. Such a sample can be collected by any suitable method. For example, for able-bodied men, the step of providing sperm can be performed by masturbation to achieve ejaculation. For men with SCI, suitable methods of achieving ejaculation include penile vibratory stimulation (PVS) and electroejaculation (EEJ). Such methods are described in more detail in the Examples sections presented below. For non-human mammals, conventional techniques for obtaining semen samples are known in the art of animal husbandry.

The biological sample containing sperm can vary according to the site selected for contacting the sperm with the agent. In some embodiments, the fluid component of the sample is that of the ejaculate, i.e., the seminal plasma. As an example, a semen sample may obtained from the subject and contacted with an agent in vitro. In other aspects, e.g., if the inhibition of the cytokine is performed within the reproductive tract of the male subject, the fluid can be that fluid produced by one or more of the tissues lining the male reproductive tract, including the seminiferous tubules, epididymis, vas deferens, prostate, seminal vesicles and urethra. In yet other embodiments, it is envisioned that the agent may be contacted with sperm that have been deposited in the female reproductive tract. In that case, the fluid can include any fluid produced by any tissue of the female reproductive tract, including fluid produced by any of the ovaries, fallopian tubes, uterus, cervix or vagina. The fluid in the female reproductive tract can also be deposited semen, or an admixture of semen and any other fluid present in the female reproductive tract.

The method includes a step of contacting the biological sample with an agent that inactivates or reduces the biological activity of at least one cytokine present in the sample. Any cytokine that results in reduction in sperm motility can be targeted by the methods of the invention. Among the preferred cytokines are those that have been shown to be secreted by leukocytes, such as activated T lymphocytes, that are present in abnormally high numbers in the seminal plasma of human subjects with leukocytospermia (Basu et al., 2002). Preferred targets of cytokine inactivation in subjects suffering from SCI include inflammatory cytokines such as TNFα, IL1β and IL6, as disclosed herein.

The method utilizes an agent that inactivates or reduces the biological activity of a cytokine in the fluid that contacts the sperm. Numerous agents can be used to inactivate a cytokine in a fluid, or alternatively to modulate expression of a cytokine in a tissue or cell known to secrete the cytokine into the extracellular medium. Any of these methods that is suitable for the particular purpose may be employed. Typical agents for inactivating cytokines in a fluid medium include proteins such as antibodies. Agents useful to modulate expression of a cytokine by a cytokine-producing cell include proteins, nucleic acids, and small organic or inorganic molecules. Examples of nucleic acids useful to modulate expression of cytokines include antisense oligonucleotides and ribozymes.

Examples of proteins that can inactivate or reduce the biological activity of TNFα, IL1β or IL6 are antibodies that specifically bind TNFα, IL1β and IL6, respectively. Such antibodies can be used to interfere with the interaction of TNFα, IL1β and IL6 protein and other molecules that bind TNFα, IL1β and IL6 protein, and are responsible for reduced sperm motility.

In an example of the use of this method, monoclonal antibodies directed against TNFα, IL1β and IL6 were added directly to semen of men with SCI, with resultant improvement in sperm motility (See Example 2 and Table 3 for details.) When added to samples of sperm from normal subjects, seminal plasma from men with SCI, treated with monoclonal antibodies to neutralize activity of specific inflammatory cytokines, had no effect on normal sperm motility.

### EXAMPLES

The following examples serves to illustrate the invention without limiting it thereby. It will be understood that variations and modifications can be made without departing from the spirit and scope of the invention.

### Example 1

### Detection and Immunophenotypic Analysis of Leukocytes in Semen Samples

Methods

Subjects. All subjects (n=17) were men with SCI who were participants in the Male Fertility Research Program of the Miami Project to Cure Paralysis at the University of Miami, School of Medicine, Miami, Fla. The mean age of subjects was 35.2±2.2 years (range 21 to 43 years). All subjects were past the acute phase of injury, and their mean years post-injury was 13.3±3.8 years (range 3 to 32 years). Levels of injury as assessed by the University of Miami Neurospinal Index (Klose et al, 1980) were C5 to C6 in five subjects, T1 to T7 in six subjects and T8 to T12 in six subjects. Each subject had undergone at least four ejaculations spaced 4 to 8 weeks apart prior to semen collection for this study. All subjects were in good health and did not have any condition, other than SCI, known to interfere with fertility.

Semen collection and analysis. Only antegrade semen (i.e., no retrograde semen) was collected from subjects by the standard method of penile vibratory stimulation (Brackett et al, 2000) and semen analysis was performed according to World Health Organization criteria (1999). Each semen specimen was first allowed to liquefy at room temperature. Sperm parameters were assessed by placing 6 μl of the semen specimen on a disposable semen analysis chamber (Cell-Vu, Fertility Technologies, Natick, Mass.). Sperm motility was evaluated in subjects before and after exposure to monoclonal antibodies. The study was evaluator-blind, where the operator did not know the treatment conditions of the specimen being evaluated. Sperm motility was calculated by adding the percent of rapid and sluggish sperm with forward movement. The same operator evaluated all specimens.

Specific monoclonal antibodies. Specific monoclonal antibodies to human cytokine interleukin 1 beta (IL1β), interleukin 6 (IL6) and tumor necrosis factor alpha (TNFα) were used to neutralize cytokine activity in the seminal plasma. These agents were selected according our previous finding that concentrations of these specific cytokines are elevated in the seminal plasma of patients affected by SCI (Basu et al, in press).

Monoclonal antibodies to human IL1β, IL6 and TNFα (R&D Systems, MN, catalog numbers DLB50, D6050 and DTA50, respectively) were reconstituted in sterile phosphate buffered saline (PBS), pH 7.2 and a stock solution of 500 μg/ml of each cytokine was prepared. The stock solution was aliquoted in 200 μl in sterile microfuge tubes and frozen at −20° C. As needed, the stock solution was further diluted to 10 μl/ml and 1 μl/ml with PBS and frozen at −20° C. until used.

Experimental design. Each semen specimen was separated into eight 50 μl aliquots, and specific monoclonal antibodies to IL1β, IL6 and TNFα were added singly and in all possible combinations. Doses were adjusted according ED50 information provided by the manufacturers. There were eight different treatment groups for each specimen.

1. 50 μl semen+10 μl of buffer (untreated control)

2. 50 μl semen+10 μl of mAb TNFα 1 μg/ml

3. 50 μl semen+10 μl of mAb IL1β 1 μg/ml

4. 50 μl semen+10 μl of mAb IL61 μg/ml

5. 50 μl semen+10 μl of mAb TNFα 1 μg/ml+10 μl of mAb IL1β 1 μg/ml

6. 50 μl semen+10 μl of mAb TNFα 1 μg/ml 50 μl semen+10 μl of mAb IL6 1 μg/ml

7. 50 μl semen+10 μl of mAb IL1β 1 μg/ml+10 μl of mAb IL61 μg/ml

8. 50 μl semen+10 μl of mAb TNFα 1 μg/ml+10 μl of mAb IL1β 1 μg/ml+10 μl of mAb IL6 1 μg/ml

After 1-2 hours incubation at room temperature, sperm motility was analyzed in each preparation. Mean sperm motility in untreated versus treated preparations was compared by analysis of variance.

Results

Similar to previously published reports of semen quality obtained by penile vibratory stimulation (Brackett et al, 1997), the mean sperm concentration±standard error of the mean of SCI subjects was 77.6±11.5×10⁶/ml (millions of sperm per milliliter of ejaculate), and the mean sperm motility was 20.1±3.1% (percentage of sperm with forward progression). Table I shows the mean sperm motility in the eight treatment groups. Sperm motility increased in all groups treated with mAb, however, statistical significance was achieved only in the group receiving mAb against all three cytokines (group 8).

TABLE 1
SPERM MOTILITY AFTER TREATMENT

| | 1 | | | | | | | |
|---|---|---|---|---|---|---|---|---|
| n = 17 | (untreated) | 2 | 3 | 4 | 5 | 6 | 7 | 8 |
| % motility | 20.1 ± 3.1 | 24.2 ± 4.2 | 26.6 ± 4.9 | 27.4 ± 5.3 | 27.2 ± 5.3 | 28.1 ± 5.4 | 29.5 ± 5.9 | 36.0 ± 4.9 |
| Significance | NA | NS | NS | NS | NS | NS | NS | p < 0.02 |

Legend for Table 1:
% motility = mean percent of motile sperm ± SEM
NA = not applicable
NS = not significant
Group 1: untreated (control group)
Group 2: mAb to TNFα
Group 3: mAb to IL1β
Group 4: mAb to IL6
Group 5: mAb to TNFα + IL1β
Group 6: mAb to TNFα + IL6
Group 7: mAb to IL1β + IL6
Group 8: mAb to all three cytokines

### Example 2

### Cytokine Receptor Blockers Improve Sperm Motility

Materials and Methods

Subjects. Subjects were eleven men with traumatic spinal cord injury and five non-SCI, healthy control subjects.

Semen collection. Semen was collected from SCI subjects by the standard method of penile vibratory stimulation. Only antegrade fractions were used in the study. Retrograde ejaculates and electroejaculates were not used in this study because these procedures have been shown to alter semen quality. Control subjects collected their semen by masturbation following 3-7 days of abstinence from ejaculation.

Semen analysis. Each specimen was allowed to liquefy (20-30 minutes) at room temperature, then placed on a disposable semen analysis chamber (Cell-Vu, Fertility Technologies, Natick, Mass) Semen analysis was performed by standard methods according to World Health Organization criteria (1999). SCI subjects with a sperm motility≦40% and control subjects a sperm motility≧50% were selected for the study. The sperm motility in selected subjects is shown in table 2.

TABLE 2
Sperm motility in SCI and control subjects
before treatment with specific receptor blockers.

| | Controls | SCI |
|---|---|---|
| Sperm Motility % | (n) | (n) |
| 0-5 | 0 | 2 |
| 6-19 | 0 | 7 |
| 20-40 | 0 | 2 |
| >50 | 5 | 0 |
| TOTAL | 5 | 11 |

Cytokine receptor blockers. The following specific blockers were used to interfere with cytokine receptors: monoclonal anti-interleukin-6 receptor antibody from mouse (mAB to IL-6 R, Sigma-Aldrich, catalog # 10649), -recombinant human soluble TNF receptor type 1 (sTNF RI, R&D Systems, catalog # 636-R1) and recombinant human soluble IL-1 receptor type II, (sIL-1 RII, R&D Systems, catalog # 263-2R). “In vitro” doses were adjusted based on ED50 information provided by the manufacturer.

Experimental Design. Sperm was separated from the seminal plasma by centrifugation at 2,000 rpm for 10 min. For each subject, seven aliquots were prepared, each containing 5,000 sperm suspended in 50 μl seminal plasma. The aliquots were treated as follows to provide all individual and combined anti-cytokine-receptor complex treatments:

Group 1: Sperm aliquot treated with 1.8 ng of recombinant human sTNF RI/μl semen.

Group 2: Sperm aliquot treated with 50 ng of recombinant human sIL-1 receptor type II/μl semen.

Group 3: Sperm aliquot treated with 2 ng of monoclonal anti-IL-6 receptor antibody/μl semen

Group 4: Sperm aliquot treated with 1.8 ng sTNF RI+50 ng sIL-1 RII/μl semen.

Group 5: Sperm aliquot treated with 1.8 ng sTNF RI+2 ng mAB to IL-6 R/μl semen.

Group 6: Sperm aliquot treated with 50 ng sIL-1 RII+2 ng mAB to IL-6 R/μl semen.

Group 7: Sperm aliquot treated with 1.8 ng sTNF RI+50 ng sIL-1 RII+2 ng mAB to IL-6 R/μl semen.

The sample plus blocker preparations were incubated at room temperature for one hour. Since protein-ligand binding is a reversible phenomenon, we evaluated sperm motility each 20 min during the incubation period. The highest sperm motility obtained during the 1 hour incubation was designated as the “treated” motility.

Cytokine Determination. The protease inhibitor phenylmethylsulfonylfluoride (PMSF; 0.5 mM)) was added to the remaining seminal plasma which was then stored at −80° C. until used for cytokine determination by Enzyme-linked Immunosorbent Assay (ELISA). IL1, IL6, and TNFα were measured in the seminal plasma of SCI and control subjects using ELISA kits. Seminal plasma samples were added to the wells of microtitration plates pre-coated with a specific anti-cytokine monoclonal antibody. After incubation at room temperature for 2 hours, the unbound components were removed by washing. The second anti-human cytokine biotin-conjugated antibodies were added and incubated for 2 hours at room temperature. After washing the wells, streptavidin-horseradish peroxidase (polyconjugated) was added and incubated for 20 min at room temperature. Finally, substrate was added, color was developed for 15 min and the reaction was stopped with 2N sulfuric acid. Absorbance was measured at 450 nm with an ELISA reader. Samples were assayed twice in duplicate.

Evaluation of response to cytokine receptor blocker treatments. Response to receptor blocker treatments was evaluated by comparing pre-treatment sperm motility to post-treatment sperm motility.

Results

Table 3 shows cytokine concentrations in seminal plasma of control subjects and the effect of receptor blockers on sperm motility in this group. TNFα was undetectable by ELISA in all cases (100%), and IL1β and IL6 concentrations were within normal values. Sperm motility was not affected by exposure to receptor-blocker treatments.

TABLE 3
Cytokine concentrations (pg/ml) in seminal plasma of
control subjects and sperm motility before and after
blocker treatments (%)

| Subject | | | | | |
|---|---|---|---|---|---|
| (n) | TNF-alpha | IL-6 | IL-1beta | SM-pre | SM-post |
| 1 | — | 3.6 | 5.1 | 52 | 52 |
| 1 | — | 8.2 | 4 | 56 | 54 |
| 1 | — | 5.7 | 3.6 | 51 | 54 |
| 1 | — | 12.1 | 5 | 52 | 55 |
| 1 | — | 9 | 2.1 | 58 | 54 |

SM-pre: sperm motility before treatment with specific cytokine receptor blockers

SM-post: sperm motility after treatment with specific cytokine receptor blockers

Expected seminal plasma values for healthy men:

TNF alpha: less than 5 pg/ml

IL-6: between 4-15 pg/ml

IL-1beta: between 1-5 pg/ml

Results from SCI patients are shown in Table 4. Post-treatment sperm motility was higher than pre-treatment sperm motility in all patients, but improvement was less pronounced in Subjects 2, 5 and 6. In Subject 2, cytokine concentrations were nearly normal. In Subject 5, TNFα was undetectable, and IL6 and IL1β concentrations were not greatly elevated. Subject 6, pre-treatment sperm motility was approaching normal (normal is ≧50%).

TABLE 4
Cytokine concentrations in seminal plasma and
sperm motility in 11 men with spinal cord injury
before and after treatment with all three receptor blockers

| SCI | TNFα | IL6 | IL1β | SM-pre | SM-post |
|---|---|---|---|---|---|
| Subject | (pg/ml) | (pg/ml) | (pg/ml) | % | % |
| 1 | 59.2 | 132.0 | 43 | 33 | 52 |
| 2 | 3.0 | 13 | 6 | 18 | 20 |
| 3 | 244.4 | 11 | 36 | 19 | 49 |
| 4 | 3.6 | 428 | 44 | 19 | 34 |
| 5 | — | 56.5 | 33 | 15 | 21 |
| 6 | 14.4 | 103.2 | — | 41 | 49 |
| 7 | 245 | 101.4 | 11.3 | 11 | 34 |
| 8 | 58.3 | 623 | 50.2 | 0 | 7 |
| 9 | 104.2 | 320.5 | — | 17 | 42 |
| 10 | 3.0 | 428 | 28.3 | 15 | 34 |
| 11 | 247.0 | 58 | 3.8 | 4 | 29 |

SM-pre: sperm motility before treatment with specific cytokine receptor blockers
SM-post: sperm motility after treatment with specific cytokine receptor blockers

Table 5 shows mean sperm motility in specimens from men with spinal cord injury before and after treatment with receptor blockers alone and in all possible combinations.

TABLE 5
SCI SEMEN SAMPLES TREATED WITH RECEPTOR BLOCKERS
TO TNFα, IL1β AND IL6.
N = 11 cases.

| | Group | | | | | | | |
|---|---|---|---|---|---|---|---|---|
| | 1 | | | | | | | |
| | (untreated) | 2 | 3 | 4 | 5 | 6 | 7 | 8 |
| % motility | 17.4 ± 3.4 | 19.7 ± 3.3 | 24.9 ± 4.2 | 21 ± 3 | 23.6 ± 3.7 | 22.8 ± 3.5 | 24.9 ± 3.8 | 33.7 ± 4.2 |
| Significance | NA | NS | NS | NS | NS | NS | NS | p < 0.03 |

Results are mean ± standard error of the mean.
NA = not applicable
NS = not significant
Group 1: untreated
Group 2: Blocker to TNFα receptor
Group 3: Blocker to IL1β receptor
Group 4: Blocker to IL6 receptor
Group 5: Blockers to TNFα + IL1β receptors
Group 6: Blockers to TNFα + IL6 receptors
Group 7: Blockers to IL1β + IL6 receptors
Group 8: Blockers to all three receptors

| | Controls | SCI |
|---|---|---|
| Sperm Motility % | (n) | (n) |
| 0-5 | 0 | 2 |
| 6-19 | 0 | 7 |
| 20-40 | 0 | 2 |
| >50 | 5 | 0 |
| TOTAL | 5 | 11 |

### Literature Cited

Aird I A, Vince G S, Bates M D, Johnson P N, Lewis-Jones D I: Leukocytes in semen from men with spinal cord injuries. Fertil Steril 1999; 72: 97-103.

Aitken R J, West K, Buckingham D: Leukocytic infiltration into human ejaculate and its association with semen quality, oxidative stress and sperm function. J Androl 1994; 15: 343-352.

Aitken R J, Irvine D S, Wu F C: Prospective analysis of sperm oocyte fusion and reactive oxygen species generation as criteria for the diagnosis of infertility. Am J Obstet Gynecol 1991 164: 542-551.

Basu S, Lynne C M, Ruiz P, Aballa T C, Ferrell S M, Brackett N L: Cytofluographic identification of activated T-cell subpopulations in the semen of men with spinal cord injuries. J Androl 2002; 23:551-556.

Brackett N L, Davi R C, Padron O F, Lynne C M: Seminal plasma of spinal cord injured men inhibits sperm motility of normal men. J Urol 1996a 155:1632-1635.

Brackett N L, Nash M S, Lynne C M: Male fertility following spinal cord injury: facts and fiction. Physical Therapy 1996b; 76:1221-1231.

Brackett N L: Semen retrieval by penile vibratory stimulation in men with spinal cord injury. Human Reprod Update 1999; 5: 216-222.

Brackett N L, Ead D N, Aballa T C, Ferrell S M, Lynne C M: Semen retrieval in men with spinal cord injury is improved by interrupting current delivery during electroejaculation. J Urol 2002; 167: 201-203.

de Lamirande E and Gagnon C: Human sperm hyperactivation in whole semen and its association with low superoxide scavenging capacity in seminal plasma. Fertil Steril 1993; 59:1291-1295.

Gruschwitz M S, Brezinschek R, Brezinschek H P: Cytokine levels in the seminal plasma of infertile males J Androl 1996; 17: 158-163.

Kovalski N N, de lamirande E, Gagnon C: Reactive oxygen species generated by human neutrophils inhibit sperm motility: protective effect of seminal plasma and scavengers. Fertil Steril 1992; 58:809-816.

Lisenmeyer T A & Perkash I: Infertility in men with spinal cord injury Arch Phys Med Rehabil. 1991;72:747-754.

Naz R K, Evans W: Decreased levels of interleukin 12 are not correlated with leukocyte concentration and superoxide dismutase activity in semen of infertile men Arch Androl 1998; 41:91-96.

Ohl D A, Denil J, Fitzgerald-Shelton K et al: Fertility of spinal cord males: effect of genitourinary infection and bladder management on results of electroejaculation. J Am Paraplegic Soc. 1992; 15:53-59.

Padron O F, Brackett N L, Sharma R K, Lynne C M, Thomas A J, Jr. Agarwal A. Seminal reactive oxygen species and sperm motility and morphology in men with spinal cord injury. Fertil Steril 1997; 67:1115-1120.

Rajasekaran M, Hellstrom W J, Naz R K, Sikka S C: Oxidative stress and interleukins in seminal plasma during leukocytospermia. Fertil Steril 1995; 64:166-171.

Sonksen J, Biering-Sorensen F. Fertility in men with spinal cord or cauda equina lesion. Semin. Neurol 1992;12: 106-114.

Stover S L, DeLisa J A, Whiteneck G G., eds. Spinal cord injury: clinical outcomes from the model systems, Gaithersburg, Md.; Aspen Publishers, Inc; 1995.

World Health Organization laboratory manual for examination of human semen and sperm cervical mucous interaction., Cambridge, United Kingdom; Cambridge University Press; 1999.

Wolff H, Politch J A, Martinez A, Haimovici F, Hill J A, Anderson D J Leukocytospermia is associated with poor semen quality. Fertil Steril 1990; 53: 528-536.

### Other Embodiments

While the above specification contains many specifics, these should not be construed as limitations on the scope of the invention, but rather as examples of preferred embodiments thereof. Many other variations are possible. Accordingly, the scope of the invention should be determined not by the embodiments illustrated, but by the appended claims and their legal equivalents.

What is claimed is:

1. A method of increasing motility of sperm, the method comprising the steps of:
a) providing from a subject a biological sample comprising sperm and at least one cytokine; and
b) contacting the biological sample with an agent that inactivates or reduces the biological activity of the at least one cytokine selected from the group consisting of TNFα, IL1β, and IL6.

2. The method of claim 1, wherein the subject has a condition that impairs fertility.

3. The method of claim 2, wherein the condition is leukocytospermia.

4. The method of claim 1, wherein the subject has SCI.

5. The method of claim 1 wherein the biological sample comprises a fluid produced by the male reproductive tract.

6. The method of claim 1, wherein the biological sample comprises semen.

7. The method of claim 1, wherein biological sample comprises a fluid produced by the female reproductive tract.

8. The method of claim 1, wherein the agent is an antibody that specifically binds to the at least one cytokine.

9. The method of claim 8 wherein the at least one cytokine comprises TNFα.

10. The method of claim 8 wherein the at least one cytokine comprises IL1β.

11. The method of claim 8 wherein the at least one cytokine comprises IL6.

12. The method of claim 1, wherein the agent is an antibody specifically binds to the at least one cytokine receptor selected from the group consisting of TNFα receptor, IL1β receptor, and IL6 receptor.

13. The method of claim 12, wherein the at least one cytokine receptor comprises a TNFα receptor.

14. The method of claim 12, wherein the at least one cytokine receptor comprises an IL1β receptor.

15. The method of claim 12, wherein the at least one cytokine receptor comprises an IL6 receptor.

16. The method of claim 1, wherein the agent is a soluble cytokine receptor that specifically binds to the at least one cytokine.

17. The method of claim 16, wherein the soluble cytokine receptor comprises a soluble TNFα receptor.

18. The method of claim 16, wherein the soluble cytokine receptor comprises a soluble IL1β receptor.

19. The method of claim 16, wherein the soluble cytokine receptor comprises a soluble IL6 receptor.
